(19) United States
(12) Patent Application Publication
(10) Pub. No.: US 2017/0273616 A1
(43) Pub. Date: Sep. 28, 2017

(54) SYSTEM FOR GUIDING CORRECTION OF WALKING GAIT AND CONTROL METHOD THEREOF

(71) Applicant: WEARABLE HEALTHCARE INC., Yongin-si (KR)

(72) Inventors: Hyo Sill YANG, Suwon-si (KR); Ryang Hee SOHN, Yongin-si (KR); Woo Young SIM, Suwon-si (KR)

(21) Appl. No.: 15/503,869

(22) PCT Filed: Aug. 13, 2015
(86) PCT No.: PCT/KR2015/008494

§ 371 Date: Feb. 14, 2017

(30) Foreign Application Priority Data
Aug. 14, 2014 (KR) 10-2014-0105666
Aug. 25, 2014 (KR) 10-2014-0110560

(51) Int. Cl. A61B 5/00; A61B 5/11

(52) U.S. Cl. CPC A61B 5/486; A61B 5/112; A61B 5/743; A61B 5/6807; A61B 5/1121; A61B 5/0004; A61B 2562/0219; A61B 2560/0214

(57) ABSTRACT

The present invention provides a system for guiding correction of walking gait and a control method thereof, which can make a user have fun in correction of walking gait so as to enhance a walking correction guiding effect. The control method of the system for guiding correction of walking gait includes the steps of: acquiring reference gait state information on the basis of user information; acquiring current gait state information on the basis of a gait signal received from a gait detecting device; composing a monitoring screen showing a comparison result between the reference gait state information and the current gait state information; and outputting the monitoring screen.

### TECHNICAL FIELD

The present invention relates to a system for guiding correction of walking gait and a control method thereof. More particularly, the present invention relates to a system for guiding correction of walking gait and a control method thereof, which can make a user have fun in correction of walking gait so as to enhance a walking correction guiding effect.

### BACKGROUND ART

A person's walking action is made when a supporting motion to support weight on the person's one leg and a swing motion to move the other leg while his or her weight is supported are performed in turn.

When he or she repeats the supporting motion and the swing motion to walk, it is important to walk in correct walking gait. Wrong walking gait, such as out-toed (out-toeing) gait or in-toed (in-toeing) gait, may cause unnecessary energy consumption during walking, damage joints in his or her body, especially, knee joints and ankle joints, and cause deformation in the body posture or skeleton.

As described above, when the knee joints or ankle joints are damaged, it will be more likely to be exposed to degenerative arthritis or spinal diseases. Therefore, in order to prevent such diseases and prevent deformation in posture, it is necessary to guide correction of wrong walking gait. Furthermore, it is also necessary to bring a correction effect for a beautiful and correct posture and a beauty care effect through improvement of the body posture.

### DISCLOSURE

### Technical Problem

Accordingly, the present invention has been made in view of the above-mentioned problems occurring in the prior art, and it is an object of the present invention to provide a system for guiding correction of walking gait and a control method thereof, which can make a user have fun in correction of walking gait so as to enhance a walking correction guiding effect.

### Technical Solution

To accomplish the above object, according to the present invention, there is provided a control method of a system for guiding correction of walking gait including the steps of: acquiring reference gait state information on the basis of user information; acquiring current gait state information on the basis of a gait signal received from a gait detecting device; composing a monitoring screen showing a comparison result between the reference gait state information and the current gait state information; and outputting the monitoring screen.

The user information is information inputted by a user, information recognized through analysis of an image of the user, or information transmitted or acquired from various databases.

The user information contains at least one of the user's height, weight, age and leg length, and the leg length includes the length from the hip joint to the knee joint and the length from the knee joint to the ankle joint.

The reference gait state information is calculated using the user information and an operation formula or is searched from a standard database on the basis of the user information.

The gait detecting device is wearable on the user's lower limb or is attachable to a shoe.

The monitoring screen is composed on the basis of 2D images, 3D images or real images, or the combination of the above-mentioned images.

The step of composing the monitoring screen includes the steps of: arranging objects expressing the reference gait state information, objects expressing the current gait state information and objects expressing the former gait state information; applying emphasis effects to the objects expressing the current gait state information; applying gait scores according to the current gait state information and a compensation system according to the gait scores; and displaying monitoring information, wherein the monitoring information contains at least one of a gait distance so far, an amount of physical movement so far, the gait scores, the compensation system and movement information of the lower limb joints.

The monitoring screen has an image and music, which are selected based on at least one of the user's location, current time and current season, as a background screen and a background music.

The step of arranging the objects includes the steps of: acquiring a real image through an image acquiring unit; and arranging the objects according to a footpath detected from the real image.

The control method further includes the steps of: outputting a screen of a gait analysis result including gait analysis information when the user finishes walking. The gait analysis information contains at least one of gait accuracy by steps, the entire amount of energy consumption, the entire gait distance, the entire gait time and the total number of steps.

Location information or map information are additionally combined to the gait analysis information, so that the system can be operated in combination with various contents according to the user's movement path and location, thereby providing various virtual realities and/or augmented realities.

To accomplish the above object, according to the present invention, there is provided a system for guiding correction of walking gait including: a gait detecting device for detecting a user's walking gait; and an output device for outputting a monitoring screen, which shows a comparison result between reference gait state information acquired on the basis of user information and current gait state information acquired from a gait signal of the gait detecting device.

The gait detecting device is wearable to the user's lower limb or attachable to a shoe.

The gait detecting device includes: a button unit having a power button; a detecting unit for detecting a gait signal; a control unit for processing the detected gait signal; a communication unit for transmitting the processed gait signal; and a power supply unit for supplying power to the detecting unit, the control unit and the communication unit when the power button is turned on.

The detecting unit includes at least one of a 3-axis accelerometer, a 3-axis gyroscope, a 3-axis magnetometer and an image sensor based on magnetometer.

The system for guiding correction of walking gait further includes a wireless charge device for transmitting power to the gait detecting device by wireless, wherein the gait detecting device further comprises a wireless power receiving unit for receiving power transmitted from the wireless charge device by wireless.

The wireless charge device includes: a power connection unit connected with an external power supply source; and a wireless power transmitting unit which converts DC received from the external power supply source into AC power and transmits the converted power to the gait detecting device when approach of the gait detecting device is detected.

The wireless charge device has a plate type body, and the body includes a shoe supporting part for supporting shoes to which the gait detecting devices are attached. A plurality of protrusions on which heels of the shoes are seated or markers for indicating positions where the shoes will be located are arranged on the shoe supporting part.

The reference gait state information is calculated using the user information and an operation formula or is searched from standard database information on the basis of the user information.

The reference gait state information contains at least one of dynamic gait information, static gait information and spatio-temporal gait information, and the spatio-temporal gait information contains reference stride length, reference stride time, reference stride width, and reference gait angle.

The current gait state information contains at least one of a current stride length, a current gait angle, current gait velocity, a gait distance, an amount of physical movement by walking gait, and a gait pattern.

### Advantageous Effects

As described above, the system for guiding correction of walking gait and the control method thereof according to the present invention can make the user have fun in correction of walking gait by outputting a comparison result between the reference gait state information and the user's current gait state information on a monitoring screen in real time and by applying various effects and giving opportunities to obtain various gait scores so as to make the user positively participate in correction of walking gait.

### DESCRIPTION OF DRAWINGS

FIG. 1 is a view showing a configuration of a system for guiding correction of walking gait according to a preferred embodiment of the present invention.

FIGS. 2 and 3 illustrate a state where gait detecting devices of FIG. 1 are mounted on shoes and outward forms of a charge device and outward forms of an output device according to various embodiments of the present invention.

FIG. 4 shows ranges of an average out-toeing angle and an average in-toeing angle of the ankle joint by ages while healthy persons walks.

FIG. 5 shows a configuration of the wireless charge device illustrated in FIG. 1.

FIG. 6 shows a configuration of the gait detecting device illustrated in FIG. 1.

FIG. 7 shows configurations of a wireless power transmitting unit of FIG. 5 and a wireless power receiving unit of FIG. 6.

FIG. 8 shows a configuration of the output device of FIG. 1.

FIG. 9 shows a configuration of a control unit of FIG. 8.

FIG. 10 shows an example of a user information input screen.

FIGS. 11 to 14 show examples of the monitoring screen to which emphasizing effects are applied, wherein FIG. 11 shows a gait recognition initializing screen for recognizing a user's normal gait to guide correct walking gait and FIGS. 12 to 14 shows monitoring screens on which the gait recognition initializing screen and actual gait monitoring data are superimposed.

FIGS. 15 and 16 show examples of the monitoring screen to which emphasizing effects are applied.

FIG. 17 illustrates an augmented reality-based monitoring screen.

FIG. 18 illustrates a screen showing a gait analysis result.

FIG. 19 is a flow chart showing a control method carried out by the output device out of control methods of the system for guiding correction of walking gait according to the preferred embodiment of the present invention.

### MODE FOR INVENTION

Hereinafter, exemplary embodiments of the present invention will be described with reference to the accompanying drawings. In the following description, the same elements will be designated by the same reference numerals.

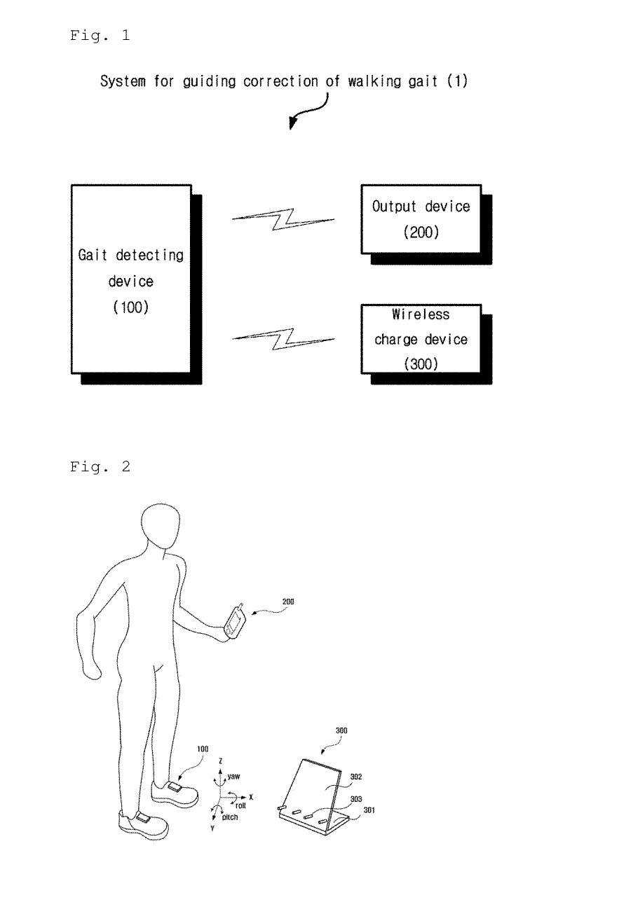

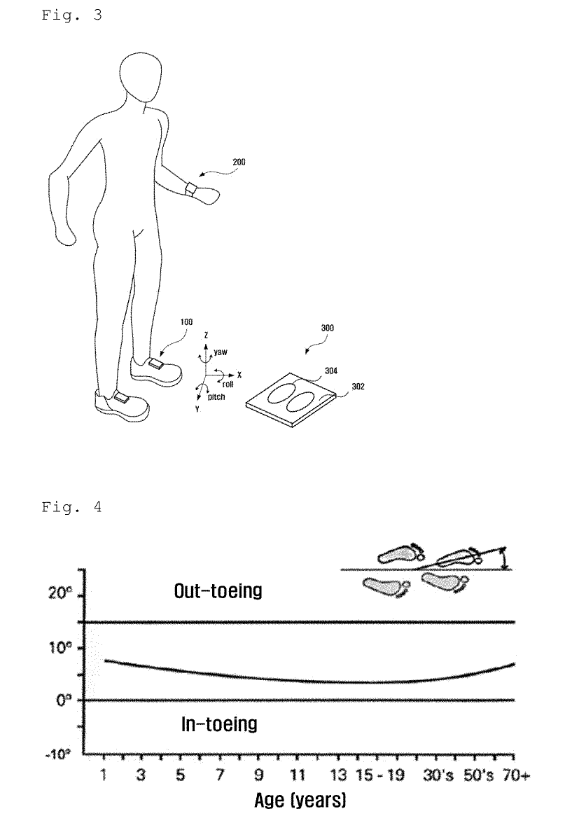

FIG. 1 is a view showing a configuration of a system for guiding correction of walking gait according to a preferred embodiment of the present invention. Additionally, FIGS. 2 and 3 illustrate a state where gait detecting devices of FIG. 1 are mounted on shoes and outward forms of a charge device and outward forms of an output device according to various embodiments of the present invention.

Referring to FIG. 1, the gait correction guiding system 1 according to the preferred embodiment of the present invention includes a wireless charge device 300, a gait detecting device 100 and an output device 200.

The wireless charge device 300 can transmit electric power to the gait detecting device 100 according to wireless power transmission techniques when the gait detecting device 100 approaches within a predetermined distance. The wireless power transmission techniques are to convert electric power into a radio frequency (RF) signal of specific frequency and transmit energy into load using electromagnetic waves generated from the RF signal. Such wireless power transmission techniques may be classified into a short-range wireless power transmission technique and a long-range wireless power transmission technique.

The short-range wireless power transmission technique may be divided into a magnetic induction (MI) type and a magnetic resonant (MR) type. The MI type is to transmit power using a magnetic field induced between a primary coil (power transmission coil) and a secondary coil (power receiving coil). The MR type is to transmit power using resonance between the primary coil (power transmission coil) connected with a source coil and the secondary coil (power receiving coil) connected with a device coil. Hereinafter, the wireless charge device 300 for transmitting power to the gait detecting device 100 according to the MI type will be described. Referring to FIG. 5, the configuration of the wireless charge device 300 will be described in detail.

Referring to FIG. 2, a body 301 and 302 of the wireless charge device 300 has a form enabling a user to put shoes thereon. For instance, the body 301 and 302 of the wire charge device 300 has a plate shape, such as a disc plate, an oval plate, a polygonal plate and so on. As shown in FIG. 2, such a body 301 and 302 includes a ground supporting part 301 which is parallel to the ground and a shoe supporting part 302 which is tilted at a predetermined angle to the ground supporting part 301 to make the shoes located. Moreover, a plurality of protrusions 303 on which heels of the shoes are seated are arranged at the lower end of the shoe supporting part 302. In this instance, when the shoes on which the gait detecting devices 100 are respectively mounted are seated on the shoe supporting part 302 and the protrusions 303, power is transmitted from the wireless charge device 300 to the gait detecting device 100.

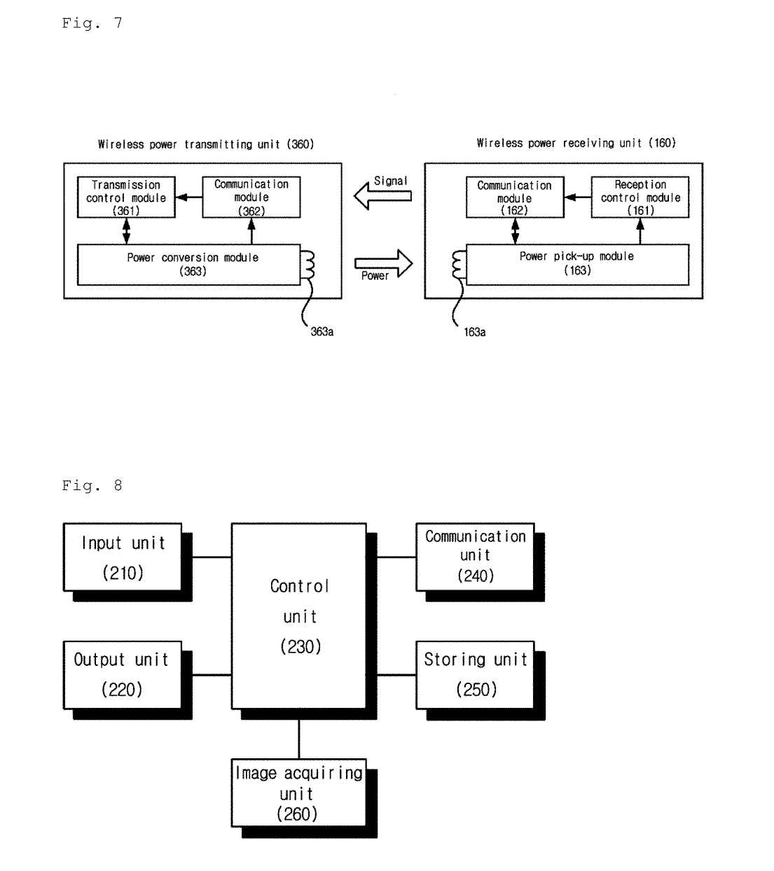

The protrusions 303 are arranged at positions to maximize power transmission efficiency from a wireless power transmitting unit (see the reference numeral 360 in FIGS. 5 and 7) of the wireless charge device 100 to a wireless power receiving unit (see the reference numeral 160 in FIGS. 6 and 7) of the gait detecting device 100. In more detail, when the center of the primary coil 363a included in the wireless power transmitting unit 360 and the center of the secondary coil 163a included in the wireless power receiving unit 160 are located on the same axis, power transmission efficiency is maximized. Therefore, arrangement of the protrusions 303 is determined in consideration of the above fact.

In the meantime, in FIG. 2, the body 301 and 302 of the wireless charge device 300 includes the ground supporting part 301 and the shoe supporting part 302, but the form of the wireless charge device 300 is not restricted to the above. According to another preferred embodiment, as shown in FIG. 3, the wireless charge device 300 may include only the shoe supporting part 302 which is parallel to the ground, and the protrusions 303 and the ground supporting part 301 may be omitted. In this instance, when the shoes on which the gait detecting devices 100 are respectively mounted are put on the shoe supporting part 302, power is transmitted from the wireless charge device 300 to the gait detecting device 100. Furthermore, markers 304 for indicating positions where the shoes or the gait detecting devices 100 will be located may be marked. The markers are indicated at spots to maximize power transmission efficiency when the shoes or the gait detecting devices 100 are put on the shoe supporting part 302.

Referring to FIG. 1, the gait detecting device 100 can sense the user's gait. For this, the gait detecting device 100 may be arranged at one of positions corresponding to the top of the foot, the inside of the ankle, the outside of the ankle, the back of the ankle. Alternatively, the gait detecting devices 100 may be arranged at two or more positions corresponding to the above-mentioned parts.

According to a preferred embodiment, as shown in FIG. 2, the gait detecting device 100 is arranged on the shoe of the user. In this instance, the gait detecting device 100 is detachably mounted on the shoe. As an example, a joining projection (not shown) may be arranged on a body (not shown) of the gait detecting device 100. Moreover, a joining groove (not shown) may be arranged in the shoe. In this instance, when the joining projection arranged on the body of the gait detecting device 100 and the joining groove arranged in the shoe are combined with each other, the gait detecting device 100 is combined to the shoe.

According to another preferred embodiment, the body of the gait detecting device 100 and the shoe may respectively have Velcro tapes. In this instance, when the Velcro tape arranged on the body of the gait detecting device 100 and the Velcro tape arranged on the shoe come into contact with each other, the gait detecting device 100 is adhered to the shoe. According to a further preferred embodiment, the body of the gait detecting device 100 may have tongs. The user can combine the gait detecting device 100 to a predetermined position of the shoe using the tongs.

According to a still further preferred embodiment, the gait detecting device 100 may be arranged on the user's lower limb. In this instance, the gait detecting device 100 is worn on the user's lower limb. For instance, the gait detecting device 100 may further include fixing means, such as a band or a belt, so as to be worn on the user's ankle. In this instance, the band or the belt are made of an elastic material for easy wearing.

As described above, the gait detecting device 100 which is mounted on the user's lower limb or the shoe senses walking gait and outputs a gait signal when the user walks. The gait signal outputted from the gait detecting device 100 is transmitted to the output device 200 according to a wire communication method or a wireless communication method. Hereinafter, the gait signal which is transmitted to the output device 200 according to the wireless communication method will be described.

Meanwhile, pairing may be carried out between the gait detecting device 100 and the output device 200 which will be described later. Pairing is the process to register device information of the gait detecting device 100 in the output device 200 and register device information of the output device 200 in the gait detecting device 100. When paring is complete, because data is transmitted and received just between the devices which completed in pairing, it can enhance security of data transmitted and received between the paired devices. However, it is not essential to carry out pairing between the gait detecting device 100 and the output device 200, and the pairing process may be omitted. Referring to FIG. 6, the configuration of the gait detecting device 100 will be described in more detail later.

The output device 200 obtains the user's current gait state information from the gait signal of the gait detecting device 100, and can gain the reference gait state information on the basis of user information. The user information may be basic information and body information. The basic information may contain a user's name, gender and age. The body information may contain at least one of a user's height, weight and leg length. For instance, the leg length is the length (hereinafter, called ‘L1’) ranging from the hip joint to the knee joint and the length (hereinafter, called ‘L2’) ranging from the knee joint to the ankle joint. Such user information may be inputted by the user or may be extracted from the user's full-length photograph or lower limb photograph at random using an image recognition program. Alternatively, the user information may be received from an external device (not shown) or an online or offline database (DB), e.g. a hospital server.

According to a preferred embodiment of the present invention, the output device 200 can compute the reference gait state information using the user information and a previously stored operation formula. According to another preferred embodiment, the output device 200 can search the reference gait state information corresponding to the user information from the standard database. The standard database means a database which stores analysis results of average walking gaits of various subject groups, such as models, soldiers, children, adults and the old. According to a further preferred embodiment, the output device 200 acquires some of the reference gait state information using the operation formula and acquires the rest of the reference gait state information by searching the standard database.

In the meantime, the reference gait state information means information which is the criterion of guiding correction of the user's walking gait, and may be static gait information, dynamic gait information and spatio-temporal gait information.

The static gait information means information related with the user's static gait. Static gait means a walking method that shift of the center of weight is small and walking speed is slow.

The dynamic gait information means information related with the user's dynamic gait. Dynamic gait means a walking method that the user walks as if he or she breaks balance and falls forward because the center of weight leaves the feet when going straight.

The spatio-temporal gait information may contain a gait velocity, a stride length, stride time, stance time, swing time and a gait angle. Here, terms will be described in brief as follows.

A step means an action carried out from the heel strike that the heel of one foot touches the ground to the heel strike that the heel of the other foot touches the ground, a step length means a horizontal distance between the heel of the front foot and the heel of the rear foot, and step time means time required for the step length.

Moreover, a stride means the action carried out from the heel strike that the heel of one foot touches the ground and the heel strike that the heel of the same foot touches the ground, a stride length means a back-and-forth distance between the two feet, and a stride width means a right-and-left distance between the two feet. Furthermore, a foot angle or an angle of gait means an angle between a gait direction and a major axis direction of the foot. Hereinafter, the stride length is called ‘reference stride length’, the stride time is called ‘reference stride time’, the stride width is called ‘reference stride width’, and the gait angle is called ‘reference gait angle’.

The reference stride length is calculated by substituting the length (L1) from the hip joint to the knee joint and the length (L2) from the knee joint to the ankle joint in the operation formula. Because the operation formula for calculating the reference stride length has been well known, description of the operation formula will be omitted.

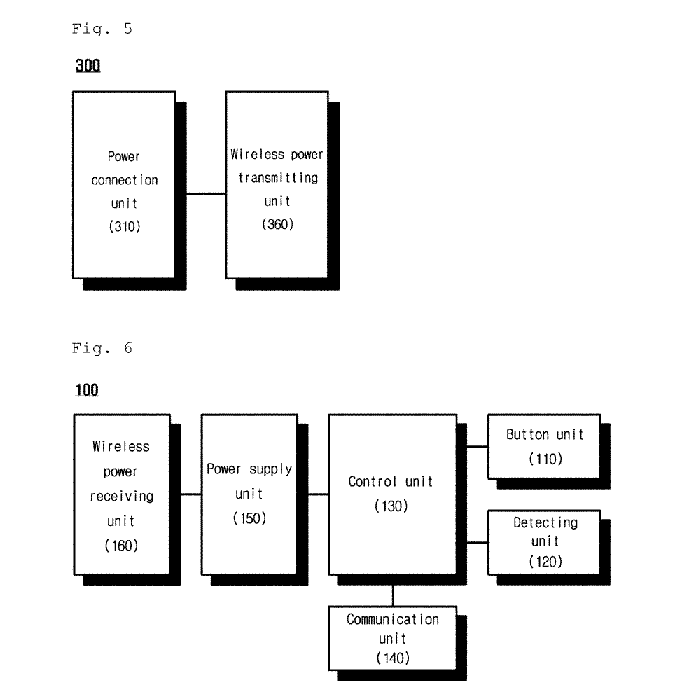

The reference gait angle may be varied according to ages. The reference gait angle is obtained by acquiring and analyzing gait data of many healthy people. FIG. 4 shows ranges of an average out-toeing angle and an average in-toeing angle of the ankle joint by ages while healthy persons walk. Referring to FIG. 4, the average out-toeing angle and the average in-toeing angle of the ankle joint of healthy persons are within the range of 0° to 15°. Therefore, it is judged whether the user's gait is out-toed (out-toeing) gait or in-toed (in-toeing) gait by comparing the illustrated range and the user's gait angle.

Referring to FIG. 1, the output device 200 can acquire the user's current gait state information on the basis of the gait signal received from the gait detecting device 100. The current gait state information may contain the current gait angle, the current gait velocity, an amount of physical movement so far according to the gait distance and walking gait, and gait pattern information.

According to a preferred embodiment, the output device 200 has a monitoring screen to show a compared result between the current gait state information and the reference gait state information, and can output the monitoring screen. The monitoring screen may be a gait state monitoring program, a program for guiding correction of walking gait or a screen provided while a game is played. The gait state monitoring program or the game for guiding correction of walking gait may be automatically carried out when the user inputs an executive command or when a gait signal is received from the gait detecting device 100.

The output device 200 can output the compared result of the current gait state information and the reference gait state information not only in a visual signal but also in an audible signal, a tactual signal, an olfactory signal or a gustatory signal or the combination of the signals. For this, the output device 200 includes an image output unit, a sound output unit, a vibration output unit, an optical output unit, a scent output unit or a taste output unit, or the combination of the units.

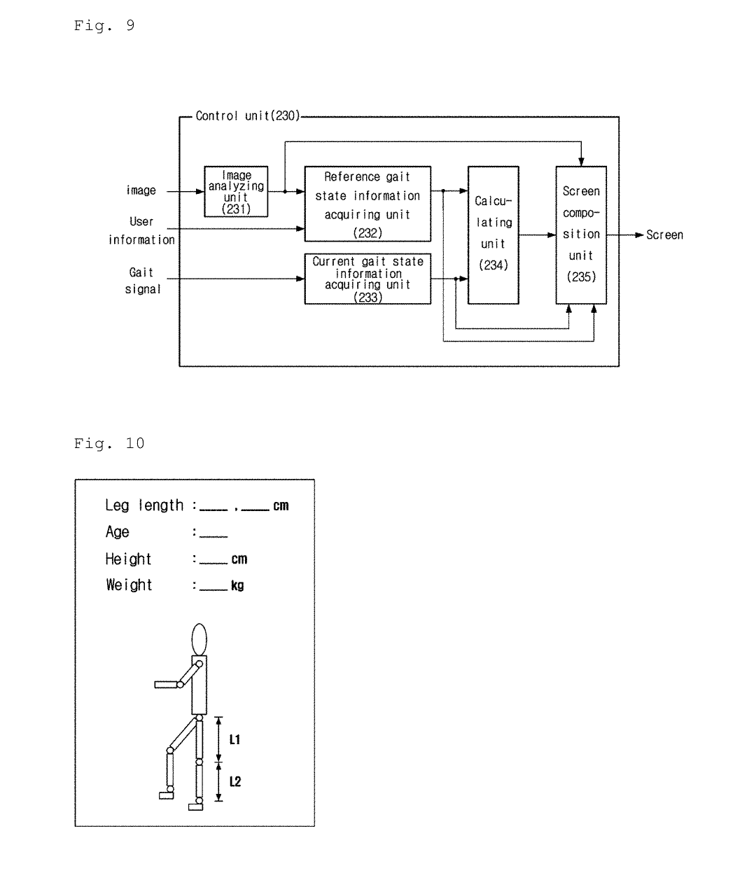

The output device 200 described above may include a wire and wireless communication device. The wire and wireless communication device may be a mobile terminal, such as a palm personal computer (Palm PC), a personal digital assistant (PDA), a wireless application protocol phone (WAP phone), a smart phone, a smart pad or a mobile play-station. The output device 200 may be a wearable device which can be worn on a part of the user's body, for instance, the head, the wrist, the finger, the arm or the waist. Referring to FIGS. 8 and 9, the configuration of the output device 200 will be described in more detail later.

FIG. 5 shows a configuration of the wireless charge device illustrated in FIG. 1. Referring to FIG. 5, the wireless charge device 300 includes a power connection unit 310 and a wireless power transmitting unit 360.

The power connection unit 310 can be connected with an external power supply source (not shown) by a cable. The power received from the external power supply source is supplied to the wireless power transmitting unit 360.

The wireless power transmitting unit 360 is operated in a pair with the wireless power receiving unit 160 of the gait detecting device 100. When approach of the gait detecting device is detected, the wireless power transmitting unit 360 transmits power received from the external power supply source to the gait detecting device 100 according to the wireless power transmission techniques.

FIG. 6 shows a configuration of the gait detecting device 100. Referring to FIG. 6, the gait detecting device 100 includes a button unit 110, a detecting unit 120, a control unit 130, a communication unit 140, a power supply unit 150 and a wireless power receiving unit 160.

The button unit 110 includes at least on button. For instance, the button unit 110 may include a power button for supplying power to the components of the gait detecting device 100. Such a power button may be, for instance, an on-off button. In FIG. 4, the gait detecting device 100 includes the button unit 110, but the button unit 110 may be omitted according to circumstances.

The detecting unit 120 can output the gait signal by detecting the user's gait when the user walks. For this, the detecting unit 120 includes a plurality of sensors. For instance, the detecting unit 120 includes a 3-axis accelerometer, a 3-axis gyroscope, a 3-axis magnetometer and an image sensor based magnetometer.

The 3-axis accelerometer can sense acceleration in the directions of x, y and z axes. The signal sensed in the 3-axis accelerometer is used to calculate the current gait state information, for instance, the current gait velocity and the gait distance.

The 3-axis gyroscope can sense angular velocity (roll, pitch and yaw) of the x, y and z axes. The signal sensed in the 3-axis gyroscope is used to calculate the current gait state information, for instance, the current gait angle.

The 3-axis magnetometer and the image sensor based on magnetometer can sense a grade to the x, y and z axes. The signals sensed in the 3-axis magnetometer and the image sensor based on magnetometer are used to calculate the current gait state information, such as the current gait angle.

The control unit 130 can connect and control the components inside the gait detecting device 100. As an example, the control unit 130 can sense on-off states of the power button of the button unit 110. As a result of the sense, if the power button is in the on state, the control unit 130 controls the components to carry out the pairing process with the output device 200. As another example, the control unit 130 can process the signal sensed by the detecting unit 120. In detail, the control unit 130 can amplify the signal sensed by the detecting unit 120 and remove noise from the amplified signal. Furthermore, the control unit 130 can convert an analog signal, from which noise is removed, into a digital signal. For this, not shown in the drawings, but the control unit 130 may include at least one of an amplifying unit, a filter unit and an A/D converting unit.

The communication unit 140 can transmit and receive data and/or control signals to and from the output device 200. For instance, the communication unit 140 can transmit and receive data and/or control signals necessary for the pairing process between the gait detecting device 100 and the output device 200. As another example, the communication unit 140 can transmit the gait signal processed by the control unit 130 of the gait detecting device 100 to the output device 200. In this instance, data, control signals and/or gait signals transmitted or received between the communication unit 140 and the output device 200 can be transmitted or received according to the wire or wireless communication techniques. As the wireless communication technique, there are Wi-Fi, Bluetooth, ZigBee and ultra-wideband.

The power supply unit 150 can supply power to the components of the gait detecting device 100. The power supply unit 150 can supply power to the components of the gait detecting device 100 when the power button of the button unit 110 is turned on. Such a power supply unit 150 may include a battery. As an example, the battery may be detachably mounted to the gait detecting device 100. As another example, hardware-wise, the battery may be formed integrally with the gait detecting device 100. In this instance, the battery is charged by power supplied through the wireless power transmission technique.

The wireless power receiving unit 160 can transmit and receive signals to and from the wireless power transmitting unit 360 of the wireless charge device 300. In detail, the wireless power receiving unit 160 responds to the signal received from the wireless power transmitting unit 360 and requests power transmission to the wireless power transmitting unit 360. As a response to the request for power transmission, when power is received from the wireless power transmitting unit 360, the wireless power receiving unit 160 supplies the received power to the power supply unit 150.

After that, the wireless power receiving unit 160 judges whether or not charging of the power supply unit 150 is finished. As a result of judgment, if charging of the power supply unit 150 is finished, the wireless power receiving unit 160 requests stop of power transmission to the wireless charge device 300. Here, FIG. 7 shows configurations of a wireless power transmitting unit of FIG. 5 and a wireless power receiving unit of FIG. 6. Referring to FIG. 7, the wireless power transmitting unit 360 includes a transmission control module 361, a communication module 362 and a power conversion module 363. The wireless power receiving unit 160 includes a reception control module 161, a communication module 162 and a power pick-up module 163.

Signals may be transmitted and received between the communication module 362 of the wireless power transmitting unit 360 and the communication module 162 of the wireless power receiving unit 160. For instance, a ping signal for searching the gait detecting device 100 may be transmitted from the wireless power transmitting unit 360 to the wireless power receiving unit 160. A response signal to the ping signal, a power transmission request signal, a power transmission stop signal and other signals may be transmitted to the wireless power transmitting unit 360 from the wireless power receiving unit 160.

The transmission control module 361 of the wireless power transmitting unit 360 controls the ping signal to be transmitted through the communication module 362. The transmission control module 361 controls the power conversion module 363 so that DC power is converted into AC power when receiving the response signal to the ping signal through the communication module 362.

The power conversion module 363 of the wireless power transmitting unit 360 converts DC power supplied from the external power supply source into AC power on the basis of the control signal of the transmission control module 361. When AC power is applied to the primary coil 363a of the power conversion module 363, electric current flows through the primary coil 363a, and an AC magnetic field is generated from the primary coil 363a by the electric current.

The AC magnetic field generated from the primary coil 363a passes through the secondary coil 163a of the power pick-up module 163, and an induced current flows in the secondary coil 163a. The power pick-up module 163 picks up AC power from the induced current flowing in the secondary coil 163a, and converts AC power into DC power. The DC power is supplied to the power supply unit 150 of the gait detecting device 100.

In the meantime, a diameter of the primary coil 363a and a diameter of the secondary coil 163a are determined in consideration of power transmission efficiency. In detail, when the center of the primary coil 363a and the center of the secondary coil 163a are located on the same axis, the diameter of the primary coil 363a and the diameter of the secondary coil 163a are equal. Moreover, when a ratio of a distance between the coils for the diameters of the coils is less than 0.1, power transmission efficiency is high. Therefore, considering the above, the diameter of the primary coil 363a and the diameter of the secondary coil 163a are determined.

FIG. 8 shows a configuration of the output device 200 of FIG. 1. Referring to FIG. 8, the output device 200 according to a preferred embodiment of the present invention includes an input unit 210, an output unit 220, a control unit 230, a communication unit 240, a storing unit 250, and an image acquiring unit 260.

The input unit 210 can receive user information or commands when the user inputs them. The user information contains the name, gender, age, height, weight, and leg length of the user. The leg length includes the length (L1) ranging from the hip joint to the knee joint and the length (L2) ranging from the knee joint to the ankle joint. The input unit 210 may be at least one of a mouse, a keyboard, a joystick, a touch pad and a touch screen, or the combination of the above-mentioned tools. In this instance, the keyboard may be realized hardware-wise or software-wise. As shown in FIG. 8, such an input unit 210 may be arranged on the output unit 200 or may be arranged on the output unit 200 and a wire and/or wireless communication device. For instance, the input unit 210 may be arranged on a remote controller.

The output unit 220 can output a command processing result in a visual signal, an audible signal, a tactual signal, an olfactory signal or a gustatory signal or the combination of the signals. For this, not shown in the drawings, but the output unit 220 includes an image output unit, a sound output unit, a vibration output unit, an optical output unit, a scent output unit or a taste output unit, or the combination of the units.

The image output unit may be a flat panel display, a flexible display or a micro display. The flat panel display or the flexible display may be an opaque display or a transparent display. The micro display is a display using an optical system and may be arranged on a head mounted display (HMD). Such an image output unit may have just the output function or may have both of the input function and the output function. For instance, if the image output unit is a touch screen, the image output unit has all of the input function and the output function.

The sound output unit may be a speaker. The optical output unit may be a light emitting diode (LED). The scent output unit includes a plurality of cartridges which have specific scent containing materials, and an air compressor which mix the materials respectively contained in the cartridges and spray the mixed materials to the outside. The taste output unit includes a plurality of cartridges which have specific taste containing materials, and an air compressor which mix the materials respectively contained in the cartridges and spray the mixed materials to the outside.

The output unit 220 can indicate a monitoring screen, which contains a comparison result between the current gait state information and the reference gait state information. The monitoring screen may be a screen provided while the gait state monitoring program or the game for guiding correction of walking gait is carried out. The monitoring screen may show 2D images, 3D images or real images, or the combination of the above-mentioned images. Kinds of the monitoring images can be determined according to predetermined values. The predetermined values may be varied by the user.

The communication unit 240 can transmit and receive data and/or control signals to and from the gait detecting device 100. For instance, the communication unit 240 can transmit and receive data and/or control signals required for the pairing process between the gait detecting device 100 and the output device 200. As another example, the communication unit 240 can receive a gait signal from the gait detecting device 100. The received gait signal is supplied to the control unit 230, which will be described later. The data and/or control signals transmitted and received between the communication unit 240 and the gait detecting device 100 can be transmitted and received according to the wire or wireless communication techniques.

The image acquiring unit 260 can acquire images. For instance, the image acquiring unit 260 can acquire images of the user's walking figure and images of the front which is in front of the user. For this, the image acquiring unit 260 includes at least one camera. The images acquired by the image acquiring unit 260 is supplied to the control unit 230, which will be described later.

The storing unit 250 can store data or algorithm required for actuation of the output device 200. For instance, the storing unit 250 can store gait state monitoring programs, data or algorithm needed for detecting a person or a footpath from the images acquired by the image acquiring unit 260, the operation formula needed for calculating the reference gait state information using the user information, the operation formula needed for calculating the current gait state information from the gait signal received from the detecting device 100, and the operation formula needed for calculating similarity between the reference gait state information and the current gait state information. Furthermore, the storing unit 250 can store graphic data required for composing the monitoring screen. Such a storing unit 250 may be a volatile memory, a non-volatile memory, a hard disc drive or an optical disc drive, or the combination of the above-mentioned tools.

The control unit 230 can acquire the reference gait state information on the basis of the user information, and acquire the current gait state information from the gait signal received from the detecting device 100. Additionally, the control unit 230 can control the components of the output device 200 so that the compared result between the reference gait state information and the current gait state information is outputted in a visual signal, an audible signal, a tactual signal, an olfactory signal or a gustatory signal or the combination of the signals.

FIG. 9 shows a configuration of the control unit 230 of FIG. 5. Referring to FIG. 9, the control unit 230 includes an image analyzing unit 231, a reference gait state information acquiring unit 232, a current gait state information acquiring unit 233, a calculating unit 234 and a screen composition unit 235.

The image analyzing unit 231 receives images. The image may be images acquired by the image acquiring unit 260 or images received from an external device. The image analyzing unit 231 analyzes the received images to recognize the user information or the footpath. The image analyzing unit 231 can detect, for instance, the user's height, leg length and weight. The detected information is supplied to the reference gait state information acquiring unit 232. If the footpath is detected from the images, the detected information is supplied to the screen composition unit 235.

The reference gait state information acquiring unit 232 acquires the reference gait state information on the basis of the user information inputted through the input unit 210 and/or the user information detected by the image analyzing unit 231. As an example, the reference gait state information acquiring unit 232 can calculate the reference gait state information using the user information and the previously stored operation formula. As another example, the reference gait state information acquiring unit 232 can search the reference gait state information corresponding to the user information from the standard database. The reference gait state information which can be acquired by the illustrative method is, for instance, reference stride length, reference gait angle, reference gait velocity, reference gait distance and reference amount of physical movement. The acquired reference gait state information is supplied to the calculating unit 234 and the screen composition unit 235.

The current gait state information acquiring unit 233 can acquire the current gait state information on the basis of the gait signal received from the gait detecting device 100. For instance, the current gait state information acquiring unit 233 can acquire at least one of current stride length, current gait angle, current gait velocity, gait distance so far and an amount of physical movement so far. The calculated current gait state information is supplied to the calculating unit 234 and the screen composition unit 235.

The calculating unit 234 calculates similarity between the reference gait state information and the current gait state information. For instance, the control unit 230 calculates similarity between the reference stride length and the current stride length, and calculates similarity between the reference gait angle the current gait angle. After that, the calculating unit 234 calculates points according to the calculated similarities. After similarity between the reference gait state information and the current gait state information is calculated while the initial program or game is carried out, a load of the calculating unit 234 is minimized to minimize power consumption and maximize usage time of the output device 200. For this, the calculating unit 234 sets a range of the gait pattern data corresponding to normal gait information, and acquires and stores data only when walking gait which is beyond the range is detected, so as to minimize use of a data storage space and optimize use of data. The calculating unit 234 can manually or automatically control at least one of a data acquisition range, the frequency of data storage and the level of sensitivity according to user settings and circumstances.

If similarity between the reference stride length and the current stride length is expressed by 0% to 100%, the corresponding range may be divided into 10 levels. If the level is high when the similarity between the reference stride length and the current stride length is high, a high point is given to a high level. For instance, if the similarity between the reference stride length and the current stride length has a value of 0% to 10%, the calculating unit 234 determines the similarity between the reference stride length and the current stride length as the level 1 and gives one point to the current stride length. If the similarity between the reference stride length and the current stride length has a value of 11% to 20%, the calculating unit 234 determines the similarity between the reference stride length and the current stride length as the level 2 and gives two points to the current stride length.

Likewise, if the similarity between the reference gait angle and the current gait angle is expressed by 0% to 100%, the corresponding range may be divided into 10 levels. If the level is high when the similarity between the reference gait angle and the current gait angle is high, a high point is given to a high level. For instance, if the similarity between the reference gait angle and the current gait angle has a value of 11% to 20%, the control unit 230 determines the similarity between the reference gait angle and the current gait angle as the level 2 and gives two points to the current gait angle.

As described above, the calculating unit 234 gives points to the current stride length and the current gait angle, and adds up the given points. According to a preferred embodiment, the calculating unit 234 simply adds up the points given to the current stride length and the current gait angle. According to another preferred embodiment, the calculating unit 234 calculates the weighted sum of the points given to the current stride length and the current gait angle. For instance, the calculating unit 234 can apply a higher weighted value to the points of the current gait angle rather than the points of the current stride length, and then, add up the values. It will be understood that the added point information is a walking gait score 48, and the walking gait score is supplied to the screen composition unit 235 which will be described later.

In the above embodiment, points are calculated in proportion to the similarity between the reference gait state information and the current gait state information. However, the method to calculate points is not restricted to the above. According to another embodiment, if the reference gait state information and the current gait state information coincide with each other, points are given, but if the reference gait state information and the current gait state information do not coincide with each other, points are deducted. It will be understood that the point information calculated by the above is the gait score 48, and the walking gait score is supplied to the screen composition unit 235 which will be described later.

The screen composition unit 235 composes a monitoring screen capable of monitoring the gait state on the basis of data supplied to at least one of the image analyzing unit 231, the reference gait state information acquiring unit 232, the current gait state information acquiring unit 233 and the calculating unit 234. In detail, the screen composition unit 235 may compose the monitoring screen by 2D images, 3D images or real images, or the combination of the above-mentioned images. The composed monitoring screen can be displayed through the image output unit of the output unit 220.

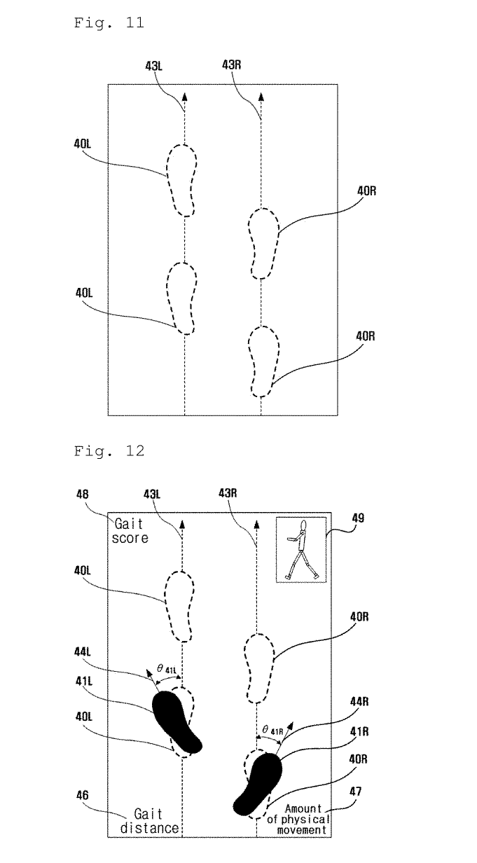

Next, referring to FIGS. 10 to 14, the monitoring screen will be described.

When a gait state monitoring program is carried out, as shown in FIG. 10, a screen for receiving the user information is displayed through the image output unit.

Referring to FIG. 10, a plurality of input fields for receiving the user information are arranged at the top of the screen. For instance, a plurality of the input fields for receiving the user's leg length, age, height and weight are arranged. Here, in connection with the leg length, an input field for the length (L1) from the hip joint to the knee joint and an input field for the length (L2) from the knee joint to the ankle joint may be arranged. FIG. 10 shows two input fields in connection with the leg length, but four input fields in connection with the leg length may be arranged so that the user can input L1 and L2 of the left leg and L1 and L2 of the right leg.

Referring to FIG. 10, a picture showing the length (L1) from the hip joint to the knee joint and the length (L2) from the knee joint to the ankle joint may be displayed at the lower end of the screen. According to another embodiment, the picture may be substituted with a text for explaining how to measure L1 and L2.

The user information is manually or automatically inputted. The user can previously set whether the user information is received manually or automatically. In case that a user information input mode is in a manual mode, as shown in FIG. 10, a screen on which empty input fields are displayed is displayed through an image display unit. In case that the user information input mode is in an automatic mode, image analysis is preceded before the screen of FIG. 10 is displayed. For instance, after the image of the user is analyzed to detect a person, the user information, such as the leg length, height and weight, based on the detected result is recognized. As described above, when the user information is recognized, the recognized information is displayed on the input fields except the age input field of FIG. 10. After that, the user finishes input of the user information by inputting his or her age in the age input field.

When the user information input is finished, the reference gait state information is acquired on the basis of the received user information. The reference gait state information is calculated using the user information and the operation formula or is acquired through search of the standard database based on the user information.

When the reference gait state information is acquired, the monitoring screen having the reference gait state information is composed and is displayed through the image output unit. The monitoring screen may show 2D images, 3D images or real images, or the combination of the above-mentioned images. Kinds of the monitoring images can be previously set by the user. The set value may be varied during gait state monitoring. The monitoring screen is set as 2D images, screens illustrated in FIGS. 11 to 14 are displayed through the image output unit.

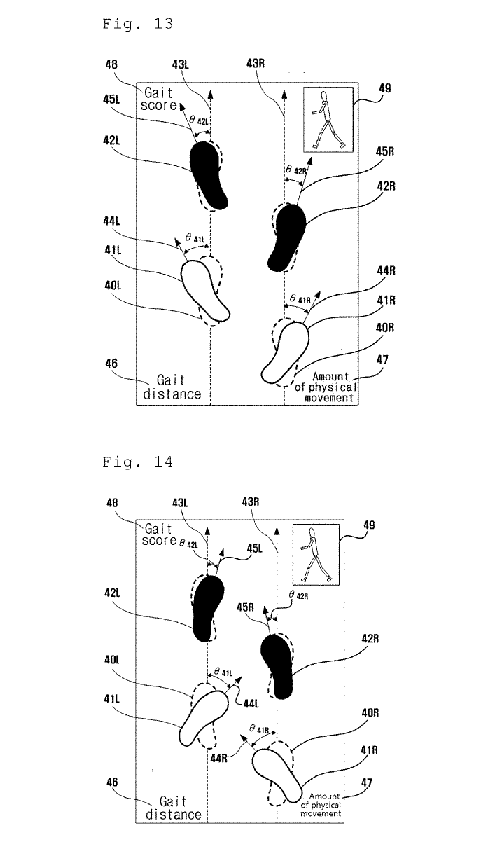

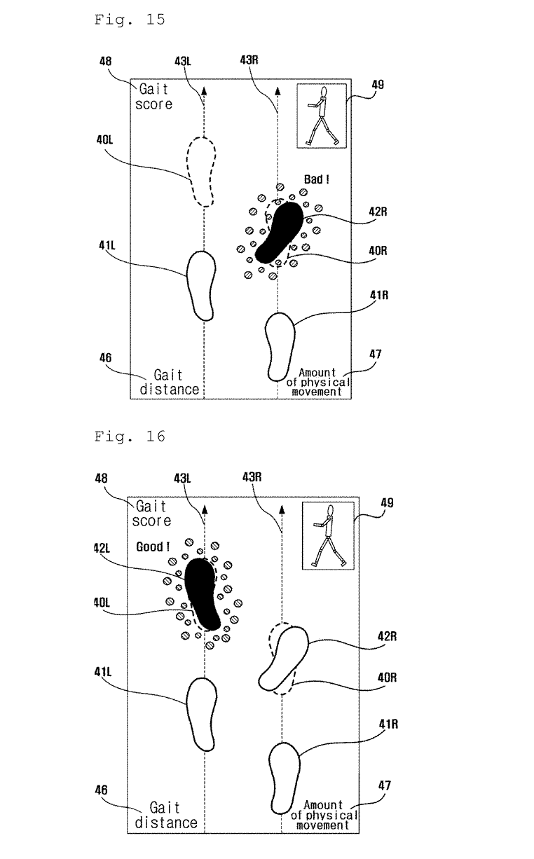

FIGS. 11 to 14 illustrate monitoring screens having 2D images. In detail, FIG. 11 shows an initial screen for user gait recognition which is displayed to obtain the user's normal gait information before the user starts walking. FIG. 12 shows a monitoring screen displayed when the user starts walking. FIG. 13 shows a monitoring screen displayed during walking when the user shows out-toed (out-toeing) gait. FIG. 14 shows a monitoring screen displayed during walking when the user shows in-toed (in-toeing) gait.

Referring to FIG. 11, a GUI object (hereinafter, called ‘object’) showing the reference gait state information is arranged in the middle of the monitoring screen. For instance, reference footprint objects 40L and 40R and reference arrow objects 43L and 43R are arranged.

The reference footprint objects 40L and 40R may be indicated by dotted lines and include the left foot's reference footprint object 40L and the right foot's reference footprint object 40R. The back-and-forth interval between the left foot's reference footprint object 40L and the right foot's reference footprint object 40R is the stride length.

The reference arrow objects 43L and 43R may be indicated by dotted lines. The direction of the reference arrow object 43L arranged along a long axis of the left foot's reference footprint objects 40L is the reference gait angle for the left foot. The direction of the arrow object 43R arranged along a long axis of the right foot's reference footprint objects 40R is the reference gait angle for the right foot.

In the above state, when the user starts to walk, as shown in FIG. 9, the monitoring screen having the reference gait state information and the current gait state information is composed and is displayed through the image output unit.

Referring to FIG. 12, objects showing the current gait state information are arranged on the monitoring screen. For instance, the first footprint objects 41L and 41R and the first arrow objects 44L and 44R are indicated by solid lines.

The first footprint objects 41L and 41R are the first left foot's footprint object 41L and the first right foot's footprint object 41R. In this instance, the back-and-forth interval between the first left foot's footprint object 41L and the first right foot's footprint object 41R is the current stride length. The direction of the first arrow object 44L arranged along a long axis of the first left foot's footprint object 41L is the current gait angle for the left foot. The direction of the first arrow object 44R arranged along a long axis of the first right foot's footprint object 41R is the current gait angle for the right foot. As described above, if the first arrow objects 44L and 44R are indicated to be overlapped relative to the first footprint objects 41L and 41R, the user can see a difference between the reference gait angle and the current gait angle at a glance.

Besides the above, an angle value θ_{41L} indicating an angle between the reference arrow object 43L and the first arrow object 44L may be expressed around the first left foot's footprint object 41L. Likewise, an angle value θ_{41R} indicating an angle between the reference arrow object 43R and the first arrow object 44R may be expressed around the first right foot's footprint object 41R.

Additionally, emphasis effect may be applied to the first footprint objects 41L and 41R to be compared with the reference footprint objects 40L and 40R. For instance, the first footprint objects 41L and 41R may be different in colors from the reference footprint objects 40L and 40R.

In the meantime, besides the current stride length and the current gait angle, other monitoring information may be displayed on the monitoring screen. The monitoring information may contain a gait distance so far, an amount of physical movement so far, gait scores so far made according to gait accuracy, and movement information of lower limb joints. Referring to FIG. 12, the gait score 48, the gait distance 46, the mount of physical movement 46 and the movement information 49 of the lower limb joints are respectively displayed at the top left corner, the bottom left corner, the bottom right corner and the top right corner.

The gait score 48 is given according to the similarity between the reference gait state information and the current gait state information. The gait score 48 may varied according to the progress of walking. If the similarity between the reference gait state information and the current gait state information is high or the reference gait state information and the current gait state information are consistent with each other, the gait score 48 is added. On the contrary, if the similarity between the reference gait state information and the current gait state information is low or the reference gait state information and the current gait state information do not match, the gait score 48 is not added or is deducted. Therefore, when the gait score 48 is deducted or added up according to accuracy of the gait state, it makes the user have fun so as to enhance the walking correction effect. According to an embodiment, the gait score 48 may have the function of money like mileage or cyber money or may be used for payment of goods which the user purchased at an enterprise registered previously. As described above, if the gait score 48 has the function of money, it motivates users to correct the walking gait state.

The amount of physical movement may be an amount of energy consumption so far. The energy consumption may be expressed by the unit of calories.

The movement information of the lower limb joints may be expressed, for instance, in 2D skeletal animation. In order to express the movement information of the lower limb joints, information on the location of the ankle joint, the location of the knee joint and the location of the hip joint is needed. Such information may be calculated on the basis of the user's leg lengths L1 and L2 and the current gait angle.

The gait score, the gait distance, the amount of physical movement and the movement information of the lower limb joints may be renewed in real time according to the progress of walking.

In the state where the monitoring screen of FIG. 12 is displayed, if the user continues walking, as shown in FIG. 13, the monitoring screen containing the reference gait state information, the former gait state information and the current gait state information is composed, and is displayed through the image output unit.

Referring to FIG. 13, because the first footprint objects 41L and 41R are objects expressing the former gait state information, the emphasis effect applied in FIG. 9 is canceled. That is, the first footprint objects 41L and 41R have the same color as the referent footprint objects 40L and 40R.

Moreover, compared with FIG. 12, in FIG. 13, second footprint objects 42L and 42R and second arrow objects 45L and 45R are additionally shown. In this instance, since the second footprint objects 42L and 42R are objects expressing the current gait state information, the emphasis effect is applied to the second footprint objects 42L and 42R differently from the reference footprint objects 40L and 40R and the first footprint objects 41L and 41R.

Furthermore, angle values θ_{42L} and 42_{R} indicating an angle between the reference arrow objects 43L and 43R and the second arrow objects 44L and 44R may be expressed around the second footprint objects 42L and 42R. Likewise, if angle values are expressed also around the second footprint objects 42L and 42R, the user can see at a glance whether or not his or her current gait state is being corrected to be closer to the reference gait state.

FIG. 14 shows a monitoring screen displaying that the user shows in-toed (in-toeing) gait. Referring to FIG. 14, compared with the gait angle θ_{41R} of the first right foot's footprint object 41R, the gait angle θ_{42R} of the second right foot's footprint object 42R is decreased. Likewise, compared with the gait angle θ_{41L} of the first left foot's footprint object 41L, the gait angle θ_{42L} of the second left foot's footprint object 42L is decreased. Therefore, the user can see that his or her current gait state is being corrected to be closer to the reference gait state.

As described above, referring to FIGS. 11 to 14, it was described that the color of the footprint objects expressing the current gait state information is displayed differently from the color of the footprint objects expressing the reference gait state information or the color of the footprint objects expressing the former gait state information. However, the emphasis effect applicable to the footprint objects expressing the current gait state information is not restricted to the above.

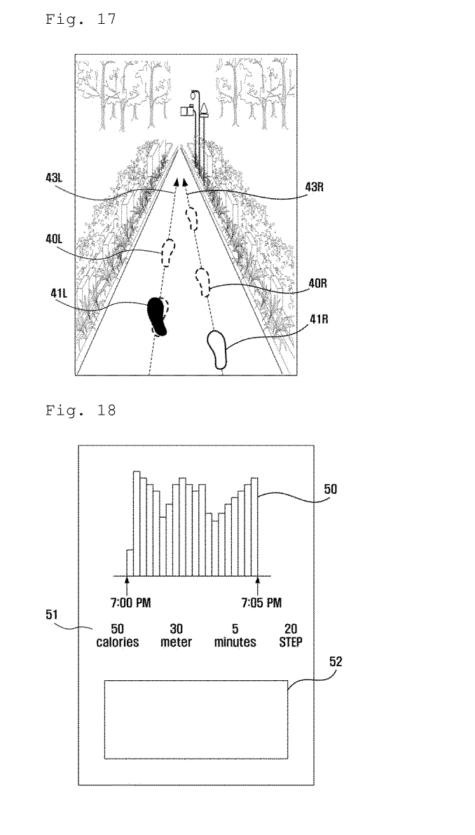

FIGS. 15 and 16 show examples of the monitoring screen to which emphasizing effects are applied.

In FIG. 15, the reference footprint objects 40L and 40R expressing the reference gait state information, the first footprint objects 41L and 41R expressing the former gait state information and the second right foot's footprint object 42R expressing the current gait state information are displayed on the monitoring screen.

Referring to FIG. 15, an effect that water drops spread around the second right foot's footprint object 42R may be added. In this instance, because the current gait angle of the second right foot's footprint object 42R and the reference gait angle of the right foot's reference footprint object 40R are not consistent with each other, the color of the water drops may be shown in color of a negative image, such as red, which is indicated by oblique lines. Additionally, a negative word, such as ‘Bad!’, may be shown around the second right foot's footprint object 42R.

In FIG. 16, the reference footprint objects 40L and 40R expressing the reference gait state information, the first footprint objects 41L and 41R and the second right foot's footprint object 42R expressing the former gait state information, and the second left foot's footprint object 42L expressing the current gait state information are displayed on the monitoring screen.

Referring to FIG. 16, because the second right foot's footprint object 42R expresses the former gait state information, the emphasis effects, such as the water drop spreading and the word, are disappeared. At the same time, the effect that water drops spread is applied around the second left foot's footprint object 42L. In this instance, since the current gait angle expressed by the second left foot's footprint object 42L and the reference gait angle expressed by the left foot's reference footprint object 40L are nearly consistent with each other, the color of the water drops may be shown in color of a positive image, such as blue, which is indicated by reversely oblique lines. In addition, a positive word, such as ‘Good!’, may be shown around the second left foot's footprint object 42L. As described above, if the emphasis effects using the water drop spreading and words are applied, the visual effect of the current gait state can be maximized.

Meanwhile, when the monitoring screen of FIG. 15 is displayed through the image output unit, an audible signal, a tactual signal, an olfactory signal or a gustatory signal with negative feelings, or the combination of the signals may be outputted. Moreover, when the monitoring screen of FIG. 16 is displayed through the image output unit, an audible signal, a tactual signal, an olfactory signal or a gustatory signal with positive feelings, or the combination of the signals may be outputted.

As described above, referring to FIGS. 11 to 13, the monitoring screens displayed through the output device 200 were described. Not shown in FIGS. 11 to 13, but the objects expressing the reference gait state information, the objects expressing the former gait state information and the objects expressing the current gait state in FIGS. 11 to 13 may be displayed to be overlapped with an image selected as a background screen. In this instance, the background screen of the monitoring screen may be an image selected on the basis of at least one of the user's location, namely, the location of the output device, present time and present season. The image used as the background screen is selected from images stored in the output device 200 or received from an image server (not shown) storing images.

According to another example, the objects expressing the reference gait state information, the objects expressing the former gait state information and the objects expressing the current gait state may be displayed to be overlapped with an actual image screen. That is, the output device 200 can display a monitoring screen based on augmented reality as shown in FIG. 17.

In order to output the monitoring screen of FIG. 17, work of taking pictures of surroundings through the image acquiring unit 260 and work of detecting a footpath from the pictures may be preceded. Furthermore, the objects are arranged according to the detected footpath to compose the monitoring screen. Differently from the monitoring screens illustrated in FIGS. 11 to 13, the monitoring screen based on the augmented reality illustrated in FIG. 17 displays the objects 40L, 40R, 41L, 41R, 43L and 43R in perspective according to the footpath.

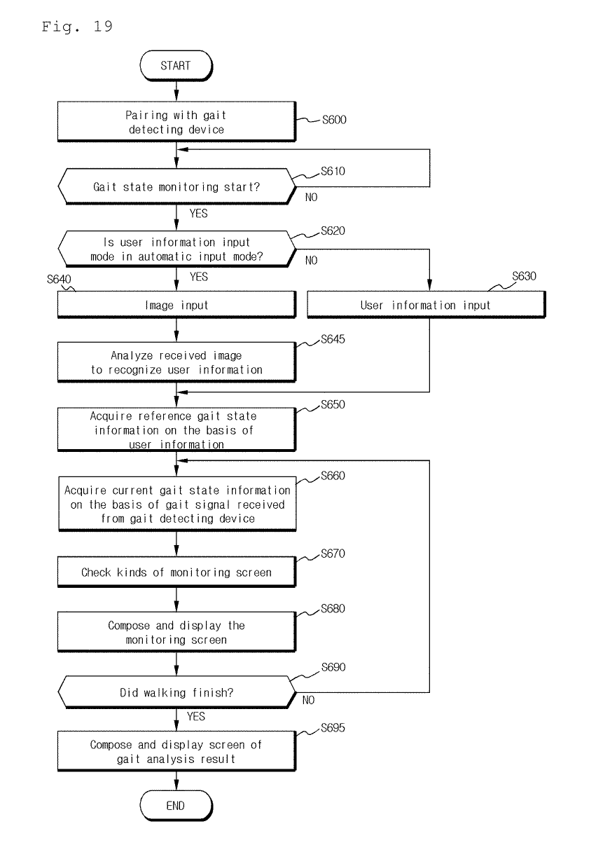

FIG. 18 illustrates a screen showing a gait analysis result. Gait analysis information may be displayed on the screen showing the gait analysis result. The gait analysis information may contain gait accuracy, the entire amount of energy consumption, the entire gait distance, the entire gait time and the total number of steps.

The gait accuracy may be, for instance, a histogram 50 expressing accuracy of each step. The horizontal axis of the histogram 50 indicates stride time, and gait start time and gait finish time are expressed on the horizontal axis. Referring to FIG. 15, the gait start time is 7 p.m. and the gait finish time is 7:05 p.m.

The vertical axis of the histogram 50 indicates accuracy of each step. Namely, the value of the vertical axis may be a similarity value between each step and the reference gait state information or a value corresponding to the similarity value. Bars on the histogram may be formed by steps. As an example, the bars by steps may be expressed in the same color. As another example, bars whose accuracy is above the reference value are different in color from bars whose accuracy is below the reference value.

Other gait analysis information 51, for instance, the entire amount of energy consumption, the entire gait distance, the entire gait time and the total number of steps, may be indicated at the lower part of the histogram 50. However, kinds of the indicated gait analysis information 51 are not restricted to the above, and may be set by the user in various ways.

A guide message window 52 may be arranged on the bottom of the screen. The guide message window 52 may show a simple guide message of the gait analysis result. For instance, a guide message to inform whether gait accuracy is high or low may be displayed.

Besides above, the guide message window 52 may show a guide message to inform whether or not the entire amount of energy consumption, the entire gait distance, the entire gait time and the total number of steps reach target values. The target values may be automatically calculated by the output device 200 in consideration of the user information or may be directly set by the user.

Location information or map information may be additionally combined to the gait analysis information, so that the system can be operated in combination with various contents according to the user's movement path and location. As a result, the system according to the present invention can provide various virtual realities and/or augmented realities.

FIG. 19 is a flow chart showing a control method carried out by the output device 200 out of control methods of the system 1 for guiding correction of walking gait according to the preferred embodiment of the present invention.

First, the control unit 230 of the output device 200 carries out pairing with the gait detecting device 100 (S600). Pairing means to register device information of the output device 200 to the gait detecting device 100 and register device information of the gait detecting device 100 to the output device 200. The step S600 includes the steps of: searching the gait detecting device 100 by the output device 200; transmitting a pairing request signal to the searched gait detecting device 100; receiving a response signal having the device information of the gait detecting device 100; and storing the device information of the gait detecting device 100.

After that, the control unit 230 decides whether or not gait state monitoring is started (S610). For instance, if the gait state monitoring program is executed, it is decided that gait state monitoring is started. The gait state monitoring program is executed when a command is inputted through the input unit 210 or when a gait signal is received from the gait detecting device 100.

When gait state monitoring starts, the control unit 230 decides whether or not the user information input mode is in the automatic input mode (S620). The user information input mode may be previously set by the user.

As a result of decision in the step S620, if the user information input mode is not in the automatic input mode, namely, if it is in the manual input mode, the control unit 230 receives the user information from the user (S630). In order to receive the user information, the screen composition unit 235 of the control unit 230 composes the screen for receiving the user information, and displays the screen through the image output unit. For instance, the screen composition unit 235 composes the screen as illustrated in FIG. 10, and displays the screen through the image output unit. When the screen of FIG. 10 is displayed, the user inputs the user information, such as leg length, age, height and weight, in the input fields by manipulating the input unit 210.

As a result of decision in the step S620, if the user information input mode is in the automatic input mode, the image analyzing unit 231 of the control unit 230 receives an image of the user's figure (S640), and then, analyzes the received image to recognize the user information (S645).

After that, the reference gait state information acquiring unit 232 of the control unit 230 acquires the reference gait state information (S650) on the basis of the user information inputted in the step S630 or the user information recognized in the steps S640 to S645. According to an embodiment, the step S630 may include the step of calculating the reference gait state information using the user information and the previously stored operation formula. According to another embodiment, the step S630 may further include the step of searching the reference gait state information corresponding to the user information from the standard database. The reference gait state information acquired in the step S 650 contains, for instance, reference stride length, reference stride time, reference gait angle, reference gait velocity, reference gait distance and reference amount of physical movement. The acquired reference gait state information is stored in the storing unit 250.

After that, when the user starts walking, the gait detecting device 100 detects the user's gait and transmits a gait signal to the output device 200.

The current gait state information acquiring unit 233 of the control unit 230 acquires the current gait state information on the basis of the gait signal received from the gait detecting device 100 (S660). The current gait state information acquired in the step S660 contains, for instance, current stride length, current gait angle, current gait velocity, gait distance so far, an amount of physical movement so far and gait pattern information.

After that, the screen composition unit 235 of the control unit 230 plays a gait correction-related game, and checks the kind of the previously set monitoring screen (S670). For instance, if the monitoring screen is set as 2D images, the monitoring screen has an image and music, which are selected based on at least one of the user's location, namely, the location of the output device, current time and current season, as a background screen and a background music. If the monitoring screen is set as a real image screen, the monitoring screen has the real image acquired through the image acquiring unit 260 as a background screen.

After the step S670, the screen composition unit 235 of the control unit 230 composes a monitoring screen having the reference gait state information, the former gait state information and the current gait state information according to the check result, and displays the monitoring screen through the image output unit (S680). The composed monitoring screen is shown in FIGS. 13 to 16.

The step S680 of composing and displaying the monitoring screen includes the steps of: arranging objects expressing the reference gait state information; arranging objects expressing the former gait state information; arranging objects expressing the current gait state information; applying the emphasis effect to the objects; applying gait scores according to the current gait state information and a compensation system, such as points, mileages or cyber money, according to the gait scores; and displaying monitoring information.

The objects expressing the current gait state information can be emphasized in various ways. For instance, as shown in FIGS. 13 and 14, the objects 42L and 42R expressing the current gait state information are different in colors, sizes, kinds of edges or the combination of the colors, sizes and kinds of edges from the other objects 40L, 40R, 41L and 41R. Alternatively, as shown in FIGS. 15 and 16, other objects, such as water drops and/or texts, are displayed around the objects 42R (in FIGS. 15) and 42L (in FIG. 16) expressing the current gait state information. In this instance, colors and sizes of the water drops and/or texts may be expressed differently from each other according to whether the current gait state information is consistent with the reference gait state information.

In the meantime, the monitoring screen may further show at least one of the gait scores given so far according to gait accuracy, the compensation system, such as points, mileages or cyber money, according to the gait scores, gait distance, the amount of physical movement, and movement information of the lower limb joints. The screen composition unit 235 of the control unit 230 renews the information by continuously receiving the gait signal to display the information on the monitoring screen.

After that, the control unit 230 decides whether the user's walking is finished (S690). As an example, if a gait finish command is inputted, it is decided that walking is finished. As another example, if the gait signal is not received from the gait detecting device 100 during a preset period of time, it is decided that walking is finished.

As a result of decision in the step S690, if walking is not finished, the control unit 230 repeats the steps S660 to S680.

As a result of decision in the step S690, if walking is finished, the screen composition unit 690 of the control unit 230 composes a screen of the gait analysis result as illustrated in FIG. 18, and displays the screen through the image output unit. The screen of the gait analysis result may contain the gait analysis information. The gait analysis information contains, for instance, gait accuracy, the entire amount of energy consumption, the entire gait distance, the entire gait time and the total number of steps.

Referring to FIG. 19, out of the control methods of the system for guiding correction of walking gait according to the present invention, the control method carried out by the output device 200 was described. Next, the control method carried out by the wireless charge device 300 and the gait detecting device 100 will be described as follows.

First, the wireless charge device 300 detects approach of the gait detecting device 100. The step of detecting approach includes the steps of: periodically transmitting a ping signal for searching the wireless power receiving unit 160 of the gait detecting device 100 from the wireless power transmitting unit 360 of the wireless charge device 300; and deciding that the wireless power receiving unit 160 of the gait detecting device 100 approaches when a response signal is received within the previously set period of time after the ping signal is transmitted.

In detail, when a distance between the gait detecting device 100 and the wireless charge device 300 becomes shorter, the wireless power receiving unit 160 of the gait detecting device 100 receives the ping signal transmitted from the wireless power transmitting unit 360 of the wireless charge device 300. For instance, if the user wearing shoes approaches the perimeter of the wireless charge device 300 or puts his or her shoes on the shoe supporting part 302 of the wireless charge device 300, the wireless power receiving unit 160 of the gait detecting device 100 can receive the ping signal transmitted from the wireless power transmitting unit 360. The wireless power receiving unit 160 which received the ping signal transmits a response signal relative to the received ping signal to the wireless power transmitting unit 360. The wireless power transmitting unit 360 decides that the gait detecting device 100 approaches when the response signal is received within the previously set period of time.

As described above, when approach of the gait detecting device 100 is detected, the wireless charge device 300 transmits power to the detecting device 100. The power transmitting step includes: sending a power transmission request from the wireless power receiving unit 160 of the gait detecting device 100 to the wireless power transmitting unit 360; converting DC power received from an external power supply source into AC power and transmitting the converted DC power by the wireless power transmitting unit 360; sending a power transmission stop request from the wireless power receiving unit 160 of the gait detecting device 100 to the wireless power transmitting unit 360; and stopping power transmission by the wireless power transmitting unit 360.

After that, the gait detecting device 100 can be charged on the basis of power received from the wireless charge device 300. The wireless charging step includes the steps of: sending power transmitted from the wireless power transmitting unit 360 of the wireless charge device 300 to the wireless power receiving unit 160 of the gait detecting device 100; supplying the received power to the power supply unit 150; and charging the power supply unit 150 by the supplied power.

The method described in relation with the embodiment of the present invention may be implemented in a software module executed by a processor. The software module can reside in a RAM, a ROM, an EPROM, an EEPROM, a flash memory, a hard disc, a detachable disc, a CD-ROM, or a recording medium which is well known in the relevant art and is readable by a computer of an arbitrary form.

While preferred embodiments of the present invention has been particularly shown and described with reference to the accompanying drawings, it will be understood by those skilled in the art that various changes in form and details may be made therein without departing from the spirit and scope of the invention as defined by the appended claims. Therefore, the preferred embodiments should be considered in descriptive sense only and not for purposes of limitation.

1. A system for guiding correction of walking gait, comprising:
a gait detecting device for detecting a user's walking gait; and
an output device for outputting a monitoring screen, which shows a comparison result between reference gait state information acquired on the basis of user information and current gait state information acquired from a gait signal of the gait detecting device.

2. The system according to claim 1, wherein the gait detecting device is wearable to a user's lower limb or attachable to a shoe.

3. The system according to claim 1, wherein the gait detecting device comprises:
a button unit having a power button;
a detecting unit for detecting a gait signal;
a control unit for processing the detected gait signal;
a communication unit for transmitting the processed gait signal; and
a power supply unit for supplying power to the detecting unit, the control unit and the communication unit when the power button is turned on.

4. The system according to claim 3, wherein the detecting unit comprises at least one of a 3-axis accelerometer, a 3-axis gyroscope, a 3-axis magnetometer and an image sensor based on magnetometer.

5. The system according to claim 1, further comprising:
a wireless charge device for transmitting power to the gait detecting device by wireless,
wherein the gait detecting device further comprises a wireless power receiving unit for receiving power transmitted from the wireless charge device by wireless.

6. A control method of a system for guiding correction of walking gait comprising the steps of:
acquiring reference gait state information on the basis of user information;
acquiring current gait state information on the basis of a gait signal received from a gait detecting device;
composing a monitoring screen showing a comparison result between the reference gait state information and the current gait state information; and
outputting the monitoring screen.

7. The control method according to claim 6, wherein the user information is information inputted by a user, information recognized through analysis of an image of the user, or information transmitted or acquired from various databases.

8. The control method according to claim 7, wherein the user information contains at least one of basic information and body information, and the basic information contains the user's name, gender and age and the body information contains at least one of the user's height, weight and leg length, and
wherein the leg length includes the length from the hip joint to the knee joint and the length from the knee joint to the ankle joint.

9. The control method according to claim 6, wherein the reference gait state information is calculated using the user information and an operation formula or is searched from a standard database on the basis of the user information.

10. The control method according to claim 6, wherein the gait detecting device is wearable on the user's lower limb or is attachable to a shoe.

11. The control method according to claim 6, wherein the monitoring screen is composed on the basis of 2D images, 3D images or real images, or the combination of the above-mentioned images.

12. The control method according to claim 6, wherein the step of composing the monitoring screen comprises the steps of:
arranging objects expressing the reference gait state information, objects expressing the current gait state information and objects expressing the former gait state information;
applying emphasis effects to the objects expressing the current gait state information;
applying gait scores according to the current gait state information and a compensation system according to the gait scores; and
displaying monitoring information,
wherein the monitoring information contains at least one of a gait distance so far, an amount of physical movement so far, the gait scores, the compensation system and movement information of the lower limb joints.

13. The control method according to claim 12, wherein the monitoring screen has an image and music, which are selected based on at least one of the user's location, current time and current season, as a background screen and a background music.

14. The control method according to claim 12, wherein the step of arranging the objects comprises the steps of:
acquiring a real image through an image acquiring unit; and
arranging the objects according to a footpath detected from the real image.

15. The control method according to claim 6, further comprising the step of:
outputting a screen of a gait analysis result including gait analysis information when the user finishes walking,
wherein the gait analysis information contains at least one of gait accuracy by steps, the entire amount of energy consumption, the entire walking distance, the entire walking time and the total number of steps.
